SEQUENCE LISTING (2 sequences)

SEQ ID NO: 1 - DNA, 1882 bp, Homo sapiens
  CDS (319)..(1533): Gene 065775
cccacgcgtccgcccacgcgtccggttcccgcgcaggcctgggtcccggctcccacaggggactacatctccccgtctgcccacgcaggagctccctcccgaggcccccggggcgcccagagcaacctcaaacccatcccgcgcgcgaaggggacagcgcagccagggcgaaggcgagggactccgtgccttcctgctttttctcctttcttccagcaaagcctggggccgaggctactcgcggagaatcgaaaagaggcaggatgagatgaggaaaactgagtggagggaaacagcgatgatgtcaagtccctttggatgggagttggggacgtgcagtttgccagggctgcctttagttccagagacacagctgtgatcagtccccaggatcccacgcttctcatcggctcctccctgctggccacctgctcagtgcacggagacccaccaggagccaccgccgagggcctctactggaccctcaacgggcgccgcctgccccctgagctctcccgtgtactcaacgcctccaccttggctctggccctggccaacctcaatgggtccaggcagcggtcgggggacaacctcgtgtgccacgcccgtgacggcagcatcctggctggctcctgcctctatgttggcctgcccccagagaaacccgtcaacatcagctgctggtccaagaacatgaaggacttgacctgccgctggacgccaggggcccacggggagaccttcctccacaccaactactccctcaagtacaagcttaggtggtatggccaggacaacacatgtgaggagtaccacacagtggggccccactcctgccacatccccaaggacctggctctctttacgccctatgagatctgggtggaggccaccaaccgcctgggctctgcccgctccgatgtactcacgctggatatcctggatgtggtgaccacggaccccccgcccgacgtgcacgtgagccgcgtcgggggcctggaggaccagctgagcgtgcgctgggtgtcgccacccgccctcaaggatttcctctttcaagccaaataccagatccgctaccgagtggaggacagtgtggactggaaggtggtggacgatgtgagcaaccagacctcctgccgcctggccggcctgaaacccggcaccgtgtacttcgtgcaagtgcgctgcaacccctttggcatctatggctccaagaaagccgggatctggagtgagtggagccaccccacagccgcctccactccccgcagtgagcgcccgggcccgggcggcggggcgtgcgaaccgcggggcggagagccgagctcggggccggtgcggcgcgagctcaagcagttcctgggctggctcaagaagcacgcgtactgctccaacctcagcttccgcctctacgaccagtggcgagcctggatgcagaagtcgcacaagacccgcaaccagcacaggacgaggggatcctgccctcgggcagacggggcacggcgagaggtcctgccagataagctgtaggggctcaggccaccctccctgccacgtggagacgcagaggccgaacccaaactggggccacctctgtaccctcacttcagggcaccttagccaccctcagcaggagctggggtggcccctgagctccaacggccataacagctctgactcccacgtgaggccacctttgggtgcaccccagtgggtgtgtgtgtgtgtgtgagggttggttgagttgcctagaacccctgccagggctgggggtgagaaggggagtcattactccccattacctagggcccctccaaaagagtccttttaaataaatgagctatttaggtgctgtgattgtgaaaaaaaaaaaaaaaaa

SEQ ID NO: 2 - protein, 404 aa, Homo sapiens
MGVGDVQFARAAFSSRDTAVISPQDPTLLIGSSLLATCSVHGDPPGATAEGLYWTLNGRRLPPELSRVLNASTLALALANLNGSRQRSGDNLVCHARDGSILAGSCLYVGLPPEKPVNISCWSKNMKDLTCRWTPGAHGETFLHTNYSLKYKLRWYGQDNTCEEYHTVGPHSCHIPKDLALFTPYEIWVEATNRLGSARSDVLTLDILDVVTTDPPPDVHVSRVGGLEDQLSVRWVSPPALKDFLFQAKYQIRYRVEDSVDWKVVDDVSNQTSCRLAGLKPGTVYFVQVRCNPFGIYGSKKAGIWSEWSHPTAASTPRSERPGPGGGACEPRGGEPSSGPVRRELKQFLGWLKKHAYCSNLSFRLYDQWRAWMQKSHKTRNQHRTRGSCPRADGARREVLPDKL

(19) United States
(12) Patent Application Publication
(10) Pub. No.: US 2003/0079239 A1
(43) Pub. Date: Apr. 24, 2003

(54) Gene Associated with bone disorders

(76) Inventors: Neelam Jaiswal, Cincinnati, OH (US); Adam Houghton, Cincinnati, OH (US); Lawrence Mertz, Gaithersburg, MD (US); Darren Ji, Cincinnati, OH (US); Jonathan S. Cook, Cincinnati, OH (US); Douglas W. Axelrod, Cincinnati, OH (US)

Correspondence Address: MORGAN LEWIS & BOCKIUS LLP, 1111 PENNSYLVANIA AVENUE NW, WASHINGTON, DC, 20004, US

(21) Appl. No.: 10/252,958

(22) Filed: Sep. 24, 2002

Related U.S. Application Data
(60) Provisional application No. 60/324,047, filed on Sep. 24, 2001.

(51) Int. Cl. A01K 67/00; C07H 21/04; C12N 9/64; C12P 21/02; C12N 5/06; C12N 5/08

(52) U.S. Cl. 800/8; 435/69.1; 435/320.1; 435/325; 435/366; 536/23.2; 435/226

(57) ABSTRACT

The present invention relates to identifying genes that are differentially regulated or expressed in bone deposition disorders. Specifically, a novel gene has been identified as being differentially regulated during the maturation of osteoblasts and whose expression can be correlated, for example, with bone deposition disorders such as osteoporosis.

### RELATED APPLICATONS

This application claims the benefit of U.S. Provisional Application No. 60/324,047 (filed Sep. 24, 2001), which is herein incorporated by reference in its entirety.

### BACKGROUND OF THE INVENTION

Living bone tissue is continuously being replenished by the processes of resorption and deposition of bone matrix and minerals. This temporally and spatially coupled process, termed bone remodeling, is accomplished largely by two cell populations, osteoclasts and osteoblasts. The remodeling process is initiated when osteoclasts are recruited from the bone marrow or the circulation to the bone surface to remove a disk-shaped packet of bone producing an area of resorbed surface. A team of osteoblasts recruited to the resorbed bone surface from the bone marrow subsequently replaces the bone matrix and mineral. Among the pathological conditions associated with abnormal bone cell function is osteoporosis, a diseased characterized by reduced amounts of bone (osteopenia) and increased bone fragility. These changes can be the result of increased recruitment and activity of osteoclasts, in combination with reduced recruitment or activity of osteoblasts (Teitelbaum et al. (1997) J. Leukoc. Biol. 61, 381-388; Simonet et al. (1997) Cell 89, 309-319).

A significant patient population that would benefit from new therapies designed to promote bone formation or inhibit resorption are those patients suffering from osteoporosis. Clinically, osteoporosis is segregated into type I and type II. Type I osteoporosis occurs predominantly in middle aged women and is associated with estrogen loss at menopause, while osteoporosis type II is associated with advancing age. An estimated twenty to twenty-five million people are at increased risk for fracture because of site-specific bone loss. The cost of treating osteoporosis in the United States is currently estimated to be in the order of ten billion dollars per year. Demographic trends, i.e., the gradually increasing age of the United States population, suggest that these costs may increase up to three fold by the year 2020 if a safe and effective treatment is not found.

Bone resorption is initiated with the destruction of bone matrix by osteoclasts. Following this initial phase of bone destruction, or resorptive phase, formation of new bone protein matrix begins. New bone proteins are deposited, and sometime later, minerals begin to be incorporated into the newly formed matrix. The formation of bone matrix and its subsequent mineralization are exclusive functions of osteoblasts.

In theory, either decreased bone formation relative to resorption or increased bone resorption relative to formation can cause the net loss of bone in osteoporosis. Control of the rate of breakdown and synthesis of new bone tissue is critical to the integrity of the skeletal structure. When the rates become unbalanced, serious conditions may result. Although there is always a net excess of bone resorption in osteoporosis, the absolute amounts of bone formation and resorption can vary from case to case.

### SUMMARY OF THE INVENTION

Few treatments are available to modulate the formation and resorption processes of bone maintenance and development. In bone disorders such as osteoporosis, it may be useful to monitor or modify the expression levels or activities of genes involved in bone formation or resorption. The present inventors have examined cell populations comprising precursor stem cells and cell populations comprising precursor stem cells that have been induced to differentiate into osteoblasts and have discovered that the expression of previously unidentified gene changes during this differentiation process. This change in gene expression provides a useful marker for diagnostic and prognostic uses as well as a marker that can be used for drug screening and therapeutic indications.

The invention encompasses an isolated nucleic acid molecule selected from an isolated nucleic acid molecule comprising the nucleotide sequence of SEQ ID NO: 1; an isolated nucleic acid molecule encoding a polypeptide comprising the amino acid sequence of SEQ ID NO: 2; an isolated nucleic acid molecule that encodes a polypeptide fragment of at least 364 amino acids of SEQ ID NO: 2; an isolated nucleic acid molecule that exhibits least 55% identity over the entire contiguous nucleotide sequence of SEQ ID NO: 1; an isolated nucleic acid molecule that exhibits at least 83% identity over the entire contiguous sequence of nucleotides 319-1533 of SEQ ID NO: 1; and an isolated nucleic acid molecule that encodes a polypeptide that exhibits at least 90% identity over the entire contiguous amino acid sequence of SEQ ID NO: 2.

In some embodiments, the isolated nucleic acid molecule is operably linked to one or more expression control elements. The invention also includes a vector comprising an isolated nucleic acid molecule and a host cell transformed to contain the nucleic acid molecule. The host cell may be either eukaryotic or prokaryotic.

The invention also encompasses a method for producing a polypeptide comprising culturing a host cell transformed with the aforementioned nucleic acid molecule under conditions in which the polypeptide encoded by said nucleic acid molecule is expressed and the isolated polypeptide produced by this method.

The invention further encompasses an isolated polypeptide selected from the group consisting of: an isolated polypeptide comprising the amino acid sequence of SEQ ID NO: 2; an isolated polypeptide comprising a fragment of at least 364 amino acids of SEQ ID NO: 2; and an isolated polypeptide exhibiting at least about 90% identity over the entire contiguous amino acid sequence of SEQ ID NO: 2. The invention includes isolated antibodies that specifically bind to the aforementioned polypeptide. The antibodies may be either monoclonal or polyclonal.

The invention includes a method of screening for an agent that modulates the differentiation of a population of stem cells into osteoblast cells comprising exposing a population of stem cells to the agent, and measuring expression or activity of a nucleic acid molecule or a polypeptide of the invention following exposure to the agent, wherein a increase in the level of expression or activity is indicative of an agent capable of stimulating stem cells to differentiate into osteoblast cells.

The invention further includes a method of screening for an agent that increases bone density comprising exposing a population of stem cells to the agent; and measuring expression or activity of a nucleic acid molecule or a polypeptide of the invention following exposure to the agent, wherein a increase in the level of expression or activity is indicative of an agent capable increasing bone density.

In some embodiments, the invention encompasses a method of diagnosing a condition characterized by abnormal stem cell differentiation comprising detecting in a stem cell sample the level of expression or activity of a nucleic acid molecule or a polypeptide of the invention, wherein a decrease in expression or activity compared to control stem cells is indicative of a condition characterized by abnormal bone density.

In a related embodiment, the invention encompasses a method of diagnosing a condition characterized by abnormal bone density comprising detecting in a stem cell sample the level of expression or activity of a nucleic acid molecule or a polypeptide of the invention, wherein a decrease in expression or activity compared to control stem cells is indicative of a condition characterized by abnormal bone density. In a preferred embodiment, the condition is osteoporosis.

The invention also includes a non-human transgenic animal comprising a nucleic acid molecule of the invention. In a preferred embodiment, the transgenic animal is engineered to not express a protein encoded by the nucleic acid molecule of the invention.

### BRIEF DESCRIPTION OF THE DRAWINGS

FIG. 1 provides a graphical representation of the expression level of the target mRNA of SEQ ID NO: 1 in human fetal stromal cells (hFSC) as assayed using READS gel analysis in response to treatment with the osteogenic agent BMP-2 (300 ng/ml) versus untreated controls. FIG. 1A shows the effect over a time period of twenty-four days whereas FIG. 1B shows effects over a period of forty-eight hours.

FIG. 2 provides a graphical representation of the expression level of the target mRNA of SEQ ID NO: 1 in hFSC assayed using READS gel analysis in response to treatment with the osteogenic agent TGF-β1(1 ng/ml) versus untreated controls. FIG. 2A shows the effect over a time period of twenty-four days whereas FIG. 2B shows effects over a period of forty-eight hours.

FIGS. 3A and 3B provides a graphical representation of the expression level of the target mRNA of SEQ ID NO: 1 in human mesenchymal stem cells as assayed using READS gel analysis in response to treatment with osteogenic and adipogenic agents. In FIG. 3A, cells were cultured in a medium containing 10% fetal calf serum with or without dexamethasone or BMP-2 for a time period ranging from zero to one hundred sixty-eight hours (7 days). In FIG. 3B, cells were cultured under adipogenic conditions in a medium containing 10% rabbit serum with or without addition of (100 nM) dexamethasone for the same time period.

FIG. 4 shows expression levels of the target mRNA of SEQ ID NO: 1 in hFSC (A) and in hMSC (B) as assayed by quantitative RT-PCR. In FIG. 4A, cells were cultured using mineralization conditions in the absence (control, closed circles with solid line) or presence of either 1 ng/ml TGF-β1 (open squares) or 300 ng/ml of BMP-2 (open triangles) for time periods of up to 504 hours (21 days). In FIG. 4B, hMSC were cultured in the absence or presence of either (1 ng/ml) TGF-β1, (300 ng/ml) BMP-2 or (100 nM) dexamethasone for time periods up to 384 hours (16 days).

FIG. 5 shows expression levels, depicted as Ct values, of the target mRNA of SEQ ID NO: 1 in various human tissues as assayed using TaqMan quantitative RT-PCR methods. The Ct values are displayed on the y axis whereas the tissue panel utilized in the assay is provided on the x axis. Expression levels of the target mRNA in resting human fetal stromal cells (hFSC control) and human mesenchymal stem cells (hMSC control) are also provided.

FIG. 6 shows a hydrophobicity analysis of the polypeptide of SEQ ID NO: 2 using the methods of Goldman et al. and of Kyte-Doolittle.

FIG. 7 shows a Northern blot in which the expression level of SEQ ID NO: 1 was measured in several normal human tissues including brain, heart, skeletal muscle, colon, thymus, spleen, kidney, liver, small intestine, placenta, lung and in leukocytes (ClonTech human mRNA blot-H12). RNA markers are present on the left side of the blot.

FIG. 8 shows a Northern blot in which the expression level of SEQ ID NO: 1 was measured in human tissues as well as in hFSC and hMSC treated with control or osteogenic agents.

FIG. 9 shows a quantitative-RT PCR experiment where different primer sets were used that can differentiate PCR amplification between the GenBank sequence AF073515 and SEQ ID NO: 1. Both hFSC (A) and hMSC (B) were used which were stimulated with the osteogenic agents shown for 24 hours or 48 hours, respectively. Primer set A consisted of a set of PCR primers directed against the known 5′ most exon of the GenBank sequence AF073515. Primer sets B and C consisted of PCR primer sets that would specifically support the amplification of SEQ ID NO: 1 and not of AF073515. Primer set B was composed of a primer set where the downstream primer was designed at the exon/exon junction (splice site) between the 5′ novel exon in SEQ ID NO: 1 and the immediate 3′ exon, whereas the upstream primer partner consisted of unique 5′ exon sequence in SEQ ID NO: 1. Primer set C consisted of a PCR primer set specific for the unique 5′ exon in SEQ ID NO: 1. The relative expression values are provided on the ordinate axis. The expression levels for the control (no osteogenic treatment) was set to 1.0.

### DETAILED DESCRIPTION

General Description

The present invention is based in part on the identification of a new gene family that is differentially expressed in bone deposition disorders. This gene, designated 65775, corresponds to the human cDNA of SEQ ID NO: 1. Genes that encode the human protein of SEQ ID NO: 2 may also be found in other animal species, particularly mammalian species. This novel gene has been identified as being differentially regulated during the maturation of osteoblasts and its expression is correlated, for example, with bone deposition disorders such as osteoporosis.

Further, monitoring of expression may be used for disease diagnosis and may be indicative of treatment efficacy. The nucleic acid molecules of SEQ ID NO: 1 or its fragments, as well as the peptides they encode, can serve as targets for agents that can be used to modulate the activity of the proteins and nucleic acids of the invention, such as the protein having the amino acid sequence of SEQ ID NO: 2. For example, agents may be identified which bind to the proteins and nucleic acids of the invention and modulate biological processes associated with bone deposition such as differentiation of stem cells into osteoblasts.

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, the term “bone density” refers to the mass or quantity of bone tissue in a certain volume of bone.

As used herein, the term “bone deposition” refers the formation of new bone during osteogenesis.

As used herein, the term “bone resorption” refers to a decrease in bone density and/or mass. Generally, mechanisms of bone resorption include, but are not limited to, secretion of enzymes and/or acids by osteoclasts to facilitate the breakdown of bone.

As used herein, the term “osteoporosis” refers to a pathological disorder characterized by a reduction in the amount of bone mass and/or density. Osteoporosis is generally characterized by increased osteoclast activity and/or decreased osteoblast activity.

As used herein, the term “stem cell” or “mesenchymal stem cell” refers to a cell capable of differentiation into an osteoblast cell. These terms are used interchangeably throughout the specification to indicate that the cell is undifferentiated.

As used herein, the terms “stem cell differentiation” and “osteoblast differentiation” refers to the process in which a stem cell develops specialized functions during maturation into an osteoblast cell.

As used herein, the term “osteoblast” refers to a cell capable of mediating bone deposition. Osteoblasts are derived from mesenchymal stem cells of the bone marrow stroma.

As used herein, the term “osteoclast” refers to a cell capable of mediating bone resorption.

Nucleic Acid Molecules

The present invention further provides nucleic acid molecules that encode the protein having SEQ ID NO: 2 and the related proteins herein described, preferably in isolated form. As used herein, “nucleic acid” is defined as RNA or DNA that encodes a protein or peptide as defined above, is complementary to a nucleic acid sequence encoding such peptides, hybridizes to SEQ ID NO: 1 under appropriate stringency conditions, or encodes a polypeptide that shares at least about 90% sequence identity over the entire contiguous amino acid sequence of SEQ ID NO: 2. The nucleic acids of the invention further includes nucleic acid molecules that share at least 55%, preferably at least about 60%, more preferably at least about 75%, and even more preferably at least about 85% or more sequence identity over the entire contiguous nucleotide sequence of SEQ ID NO: 1. The invention further includes a nucleic acid molecule that shares at least 83% sequence identity over the entire contiguous open reading frame (nucleotides 319-1533).

Specifically contemplated are genomic DNA, cDNA, mRNA and antisense molecules, as well as nucleic acids based on alternative backbones or including alternative bases whether derived from natural sources or synthesized. Such nucleic acids, however, are defined further as being novel and unobvious over any prior art nucleic acid including that which encodes, hybridizes under appropriate stringency conditions, or is complementary to nucleic acid encoding a protein according to the present invention.

Homology or sequence identity at the nucleotide or amino acid sequence level is determined by BLAST (Basic Local Alignment Search Tool) analysis using the algorithm employed by the programs blastp, blastn, blastx, tblastn and tblastx (Altschul et al. (1997) Nucleic Acids Res. 25, 3389-3402 and Karlin et al. (1990) Proc. Natl. Acad. Sci. USA 87, 2264-2268, both fully incorporated by reference) which are tailored for sequence similarity searching. The approach used by the BLAST program is to first consider similar segments, with gaps (non-contiguous) and without gaps (contiguous), between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al (1994) Nature Genetics 6, 119-129 which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al. (1992) Proc. Natl. Acad. Sci. USA 89, 10915-10919, fully incorporated by reference), recommended for query sequences over 85 nucleotides or amino acids in length.

For blastn, the scoring matrix is set by the ratios of M (i.e., the reward score for a pair of matching residues) to N (i.e., the penalty score for mismatching residues), wherein the default values for M and N are +5 and −4, respectively. Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GA-P=8 and LEN=2.

“Stringent conditions” are those that (1) employ low ionic strength and high temperature for washing, for example, 0.015 M NaCl/0.0015 M sodium citrate/0.1% SDS at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer (pH 6.5) with 750 mM NaCl, 75 mM sodium citrate at 42° C. Another example is hybridization in 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS. A skilled artisan can readily determine and vary the stringency conditions appropriately to obtain a clear and detectable hybridization signal. Preferred nucleic acid molecules are those that hybridize under the above conditions to the complement of SEQ ID NO: 1 and which encode a functional protein. Even more preferred nucleic acid molecules are those that hybridize under the above conditions to the complement strand of the open reading frame of SEQ ID NO: 1.

As used herein, a nucleic acid molecule is said to be “isolated” when the nucleic acid molecule is substantially separated from contaminant nucleic acid molecules encoding other polypeptides.

The present invention further provides fragments of the encoding nucleic acid molecule greater than 997 nucleotides. As used herein, a fragment of an encoding nucleic acid molecule refers to a small portion of the entire protein coding sequence sharing at least 83% identity or greater. The size of the fragment will be determined by the intended use. For example, if the fragment is chosen so as to encode an active portion of the protein, the fragment will need to be large enough to encode the functional regions of the protein. For instance, fragments which encode peptides corresponding to predicted antigenic regions may be prepared. If the fragment is to be used as a nucleic acid probe or PCR primer, then the fragment length is chosen so as to obtain a relatively small number of false positives during probing or priming.

Fragments of the encoding nucleic acid molecules of the present invention (i.e., synthetic oligonucleotides) that are used as probes or specific primers for the polymerase chain reaction (PCR), or to synthesize gene sequences encoding proteins of the invention, can easily be synthesized by chemical techniques, for example, the phosphotriester method of Matteucci et al. (1981) J. Am. Chem. Soc. 103, 3185-3191 or using automated synthesis methods. In addition, larger DNA segments can readily be prepared by well known methods, such as synthesis of a group of oligonucleotides that define various modular segments of the gene, followed by ligation of oligonucleotides to build the complete modified gene. In a preferred embodiment, the nucleic acid molecule of the present invention contains a contiguous open reading frame of at least about one-thousand two-hundred and fifteen nucleotides.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can readily employ any such label to obtain labeled variants of the nucleic acid molecules of the invention. Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

Isolation of Other Related Nucleic Acid Molecules

As described above, the identification and characterization of the nucleic acid molecule having SEQ ID NO: 1 allows a skilled artisan to isolate nucleic acid molecules that encode other members of the protein family in addition to the sequences herein described.

For instance, a skilled artisan can readily use the amino acid sequence of SEQ ID NO: 2 to generate antibody probes to screen expression libraries prepared from appropriate cells. Typically, polyclonal antiserum from mammals such as rabbits immunized with the purified protein (as described below) or monoclonal antibodies can be used to probe a mammalian cDNA or genomic expression library, such as lambda gtll library, to obtain the appropriate coding sequence for other members of the protein family. The cloned cDNA sequence can be expressed as a fusion protein, expressed directly using its own control sequences, or expressed by constructions using control sequences appropriate to the particular host used for expression of the enzyme.

Alternatively, a portion of the coding sequence herein described can be synthesized and used as a probe to retrieve DNA encoding a member of the protein family from any mammalian organism. Oligomers containing approximately 18-20 nucleotides (encoding about a 6-7 amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other encoding nucleic acid molecules.

Nucleic acid molecules encoding other members of the protein family may also be identified in existing genomic or other sequence information using any available computational method, including but not limited to: PSI-BLAST (Altschul et al. (1997) Nucleic Acids Res. 25:3389-3402); PHI-BLAST (Zhang et al. (1998), Nucleic Acids Res. 26, 3986-3990), 3D-PSSM (Kelly et al. (2000) J. Mol. Biol. 299, 499-520); and other computational analysis methods (Shi et al. (1999) Biochem. Biophys. Res. Commun. 262, 132-138 and Matsunami et al. (2000) Nature 404, 601-604).

Recombinant DNA Molecules Containing a Nucleic Acid Molecule

The present invention further provides recombinant DNA molecules (rDNA) that contain a coding sequence. As used herein, a rDNA molecule is a DNA molecule that has been subjected to molecular manipulation in situ. Methods for generating rDNA molecules are well known in the art, for example, see Sambrook et al. (1989) Molecular Cloning—A Laboratory Manual, Cold Spring Harbor Laboratory Press. In the preferred rDNA molecules, a coding DNA sequence is operably linked to expression control sequences and/or vector sequences.

The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 (BioRad), pPL and pKK223 (Pharmacia).

Expression vectors compatible with eukaryotic cells, preferably those compatible with vertebrate cells, can also be used to form rDNA molecules that contain a coding sequence. Eukaryotic cell expression vectors, including viral vectors, are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies Inc.), pTDT1 (ATCC), the vector pCDM8, and the like eukaryotic expression vectors.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. (Southern et al. (1982) J. Mol. Anal. Genet. 1, 327-341). Alternatively, the selectable marker can be present on a separate plasmid, and the two vectors are introduced by co-transfection of the host cell, and selected by culturing in the appropriate drug for the selectable marker. The present invention further provides host cells transformed with a nucleic acid molecule that encodes a protein of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells available from the ATCC as CCL61, NIH Swiss mouse embryo cells (NIH-3T3) available from the ATCC as CRL 1658, baby hamster kidney cells (BHK), and the like eukaryotic tissue culture cell lines.

Any prokaryotic host can be used to express a rDNA molecule encoding a protein of the invention. The preferred prokaryotic host is E. coli.

Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed, see, for example, Cohen et al. (1972) Proc. Natl. Acad. Sci. USA 69, 2110; and Sambrook et al. (1989) Molecular Cloning—A Laboratory Manual, Cold Spring Harbor Laboratory Press. With regard to transformation of vertebrate cells with vectors containing rDNAs, electroporation, cationic lipid or salt treatment methods are typically employed, see, for example, Graham et al. (1973) Virol. 52, 456; Wigler et al. (1979) Proc. Natl. Acad. Sci. USA 76, 1373-1376.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern (1975) J. Mol. Biol. 98, 503-504 or Berent et al. (1985) Biotech. 3, 208-209 or the proteins produced from the cell assayed via an immunological method.

Production of Recombinant Proteins Using a rDNA Molecule

The present invention further provides methods for producing a protein of the invention using nucleic acid molecules herein described. In general terms, the production of a recombinant form of a protein typically involves the following steps: A nucleic acid molecule is first obtained that encodes a protein of the invention, such as a nucleic acid molecule comprising, consisting essentially of, or consisting of SEQ ID NO: 1 or nucleotides 319-1533 of SEQ ID NO: 1. If the encoding sequence is uninterrupted by introns, as is this open reading frame, it is directly suitable for expression in any host.

The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

The Protein Associated with Bone Disorders

The present invention provides isolated proteins, allelic variants of the proteins, and conservative amino acid substitutions of the protein comprising the amino acid sequence of SEQ ID NO: 2. As used herein, the “protein” or “polypeptide” refers, in part, to a protein that has the human amino acid sequence depicted in SEQ ID NO: 2. The terms also refer to naturally occurring allelic variants and proteins that have a slightly different amino acid sequence than that specifically recited above. Allelic variants, though possessing a slightly different amino acid sequence than those recited above, will still have the same or similar biological functions associated with these proteins.

As used herein, the family of proteins related to the human amino acid sequences of SEQ ID NO: 2 refers to proteins that have been isolated from organisms in addition to humans. The methods used to identify and isolate other members of the family of proteins related to these proteins are described below.

The proteins of the present invention are preferably in isolated form. As used herein, a protein is said to be isolated when physical, mechanical or chemical methods are employed to remove the protein from cellular constituents that are normally associated with the protein. A skilled artisan can readily employ standard purification methods to obtain an isolated protein.

The proteins of the present invention further include insertion, deletion or conservative amino acid substitution variants of SEQ ID NO: 2. As used herein, a conservative variant refers to alterations in the amino acid sequence that does not adversely affect the biological functions of the protein. A substitution, insertion or deletion is said to adversely affect the protein when the altered sequence prevents or disrupts a biological function associated with the protein. For example, the overall charge, structure or hydrophobic/hydrophilic properties of the protein can be altered without adversely affecting a biological activity. Accordingly, the amino acid sequence can be altered, for example to render the peptide more hydrophobic or hydrophilic, without adversely affecting the biological activities of the protein.

The present invention encompasses the allelic variants, the conservative substitution variants, and the members of the protein family, having an amino acid sequence of at least about 90% amino acid sequence identity with the entire sequence set forth in SEQ ID NO: 2, more preferably at least about about 95%, even more preferably 97% and most preferably, 99% sequence identity. Identity or homology with respect to such sequences is defined herein as the percentage of amino acid residues in the candidate sequence that are identical with the known peptides, after aligning the sequences without gaps (i.e., contiguous sequences), if necessary, to achieve the maximum percent homology, and not considering any conservative substitutions as part of the sequence identity. Fusion proteins, or N-terminal, C-terminal or internal extensions, deletions, or insertions into the peptide sequence shall not be construed as affecting homology.

The proteins of the present invention include molecules having the amino acid sequence disclosed in SEQ ID NO: 2 and fragments thereof having a consecutive sequence of at least about 364 or more amino acid residues of these proteins; amino acid sequence variants wherein one or more amino acid residues has been inserted N- or C-terminal to, or within, the disclosed coding sequence; and amino acid sequence variants of the disclosed sequence, or their fragments as defined above, that have been substituted by at least one residue. The invention includes those fragments associated with osteoporosis and other bone disorders. Such fragments, also referred to as peptides or polypeptides, may contain antigenic regions, functional regions of the protein identified as regions of the amino acid sequence which correspond to known protein domains, as well as regions of pronounced hydrophilicity. The regions are all easily identifiable by using commonly available protein sequence analysis software such as MacVector (Oxford Molecular).

Contemplated variants further include those containing predetermined mutations by, e.g., homologous recombination, site-directed or PCR mutagenesis, and the corresponding proteins of other animal species, including but not limited to rabbit, mouse, rat, porcine, bovine, ovine, equine and non-human primate species, and the alleles or other naturally occurring variants of the family of proteins; and derivatives wherein the protein has been covalently modified by substitution, chemical, enzymatic, or other appropriate means with a moiety other than a naturally occurring amino acid (for example a detectable moiety such as an enzyme or radioisotope).

The present invention further provides compositions comprising a protein or polypeptide of the invention and a diluent. Suitable diluents can be aqueous or non-aqueous solvents or a combination thereof, and can comprise additional components, for example water-soluble salts or glycerol, that contribute to the stability, solubility, activity, and/or storage of the protein or polypeptide.

As described below, members of the family of proteins can be used: (1) to identify agents which modulate the level of or at least one activity of the protein, (2) to identify binding partners for the protein, (3) as an antigen to raise polyclonal or monoclonal antibodies, (4) as a therapeutic agent or target and (5) as a diagnostic agent or marker of osteoporosis and other bone disorders.

Methods to Identify Binding Partners

Another embodiment of the present invention provides methods for use in isolating and identifying binding partners of proteins of the invention. In general, a protein of the invention is mixed with a potential binding partner or an extract or fraction of a cell under conditions that allow the association of potential binding partners with the protein of the invention. After mixing, peptides, polypeptides, proteins or other molecules that have become associated with a protein of the invention are separated from the mixture. The binding partner that bound to the protein of the invention can then be removed and further analyzed. To identify and isolate a binding partner, the entire protein, for instance a protein comprising the entire amino acid sequence of SEQ ID NO: 2 can be used. Alternatively, a fragment of the protein can be used.

As used herein, a cellular extract refers to a preparation or fraction that is made from a lysed or disrupted cell. The preferred source of cellular extracts will be cells derived from human skin tissue or the human respiratory tract or cells derived from a biopsy sample of human lung tissue in patients with allergic hypersensitivity. Alternatively, cellular extracts may be prepared from normal tissue or available cell lines, particularly granulocytic cell lines.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the protein of the invention under conditions in which association of the protein with the binding partner can occur. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density/sediment centrifugation can be used.

After removal of non-associated cellular constituents found in the extract, the binding partner can be dissociated from the complex using conventional methods. For example, dissociation can be accomplished by altering the salt concentration or pH of the mixture. To aid in separating associated binding partner pairs from the mixed extract, the protein of the invention can be immobilized on a solid support. For example, the protein can be attached to a nitrocellulose matrix or acrylic beads. Attachment of the protein to a solid support aids in separating peptide/binding partner pairs from other constituents found in the extract. The identified binding partners can be either a single protein or a complex made up of two or more proteins. Alternatively, binding partners may be identified using a Far-Western assay according to the procedures of Takayama et al. (1997) Methods Mol. Biol. 69, 171-184 or Sauder et al. (1996) J. Gen. Virol. 77, 991-996 or identified through the use of epitope tagged proteins or GST fusion proteins.

Alternatively, the nucleic acid molecules of the invention can be used in a yeast two-hybrid system. The yeast two-hybrid system has been used to identify other protein partner pairs and can readily be adapted to employ the nucleic acid molecules herein described.

Modulation of Gene Expression

Pathological processes refer to a category of biological processes that produce a deleterious effect. For example, expression of the proteins of the invention is associated with differentiation of stem cells into osteoblasts under normal conditions but in a disease state, the necessary level of expression of the proteins may not be present. Such diseases include, but are not limited to, diseases caused by an abnormal rate of osteoblast formation and subsequent activity. Decreased osteoblast activity can lead to a decrease in bone deposition with a concurrent increased osteoclast activity resulting in abnormal increase in bone resorption ultimately leading to decreased bone density.

Those skilled in the art will appreciate that a wide variety of conditions are associated with an abnormal rate of osteoblast formation leading to abnormal bone deposition or loss. Such conditions include, but are not limited to, osteoporosis, osteopenia, osteodystrophy, and various other osteopathic conditions. The methods of the present invention will be particularly useful in the treatment of conditions such as postmenopausal osteoporosis (PMO), glucocorticoid-induced osteoporosis (GIO), and male osteoporosis.

Other embodiments of the present invention allow for the treatment of other conditions that involve altered bone metabolism associated with osteoblast activity, e.g., idiopathic juvenile osteoporosis (IJO). In addition, thyroid diseases have been linked to bone loss. A decrease in bone mass has been shown in patients with thyrotoxicosis causing these individuals to be at increased risk of having fractures. These individuals also sustain fractures at an earlier age than individuals who have never been thyrotoxic.

The present invention will be useful in the treatment of abnormal bone formation or loss associated with chronic anemia. Homozygous beta-thalassemia is usually described as an example of chronic anemia predisposing to osteoporosis. Patients with thalassemia have expansion of bone marrow space with thinning of the adjacent trabeculae.

Other conditions in which the present invention will find therapeutic application are: Fanconi syndrome where osteomalacia is a common feature; fibrous dysplasia, McCune-Albright syndrome refers to patients with fibrous dysplasia with a sporadic, developmental disorder characterized by a unifocal or multifocal expanding fibrous lesion of bone-forming mesenchyme that often results in pain, fracture or deformity; osteogenesis imperfecta (OI, also called brittle bone disease) is associated with recurrent fractures and skeletal deformity, various skeletal dysplasias i.e., osteochondroplasia which is characterized by abnormal development of cartilage and/or bone and other diseases such as achodroplasia, mucopolysacchaidoses, dysostosis and ischemic bone diseases.

Specifically, the expression and activation of the proteins of the invention, such as the protein having the amino acid sequence of SEQ ID NO: 2, in mesenchymal stem cells correlated with the maturation of these cells into osteoblasts and subsequent deposition of bone. The present invention therefore includes methods for modulating expression and/or activity of the proteins of the invention to effect mesenchymal stem cell differentiation and osteoblast activity. Such methods will be useful in the treatment of disorders associated with abnormal osteoblast activity. Because osteoblast activity indirectly effects osteoclast activity via a general feedback mechanism, the invention also includes methods for modulating bone resorption associated with osteoclast activity.

Modulation of the gene, gene fragments, or the encoded protein of SEQ ID NO: 2 and fragments is useful in gene therapy to treat disorders associated with defects in the protein of the invention. In a preferred embodiment, expression is modulated to increase osteoblast activity in diseases with abnormal bone density. Expression vectors may be used to introduce the nucleic acids of the invention into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g., plasmid, retrovirus, lentivirus, adenovirus and the like, where the vectors are able to transiently or stably be maintained in the cells, usually for a period of at least about one day, more usually for a period of at least about several days to several weeks.

The proteins and nucleic acids of the invention may be introduced into tissues or host cells by any number of routes, including viral infection, microinjection, or fusion of vesicles. Jet injection may also be used for intramuscular administration, as described by Furth et al. (1992) Anal. Biochem. 205, 365-368. The DNA may be coated onto gold microparticles, and delivered intradermally by a particle bombardment device, or “gene gun” as described in the literature (see, for example, Tang et al. (1992) Nature 356, 152-154), where gold microprojectiles are coated with DNA, then bombarded into skin cells. Alternatively, the proteins of the invention may be administered in soluble form to treat a bone density disorder associated with the proteins' expression. Soluble receptors have been used to bind cytokines or other ligands to regulate their function (Thomson (1998) Cytokine Handbook, Academic Press).

Antisense molecules can be used to down-regulate expression of nucleic acids or proteins of the invention in cells. The anti-sense reagent may be antisense oligonucleotides, particularly synthetic antisense oligonucleotides having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g., by reducing the amount of mRNA available for translation, through activation of RNAse H or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about seven, usually at least about twelve, and more usually at least about twenty nucleotides in length. Typical antisense oligonucleotides are usually not more than about five-hundred, more usually not more than about fifty, and even more usually not more than about thirty-five nucleotides in length, where the length is governed by efficiency of inhibition, specificity, including absence of cross-reactivity, and the like. It has been found that short oligonucleotides, of from seven to eight bases in length, can be strong and selective inhibitors of gene expression (see Wagner et al. (1996) Nat. Biotech. 14, 840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1996) Nat. Biotech. 14, 840-844). Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

As an alternative to anti-sense inhibitors, catalytic nucleic acid compounds, e.g., ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (see, for example, WO 95/23225; Beigelman et al. (1995) Nucl. Acids Res. 23, 4434-4442). Examples of oligonucleotides with catalytic activity are described in WO 95/06764.

Agents that modulate or up- or down-regulate the expression of the protein or agents such as agonists or antagonists of at least one activity of the proteins of the invention may be used to modulate biological and pathologic processes associated with the protein's function and activity. As used herein, an agent is said to modulate a pathological process when the agent reduces the degree or severity of the process. For instance, a bone density disorder may be prevented or disease progression modulated by the administration of agents which reduce, promote or modulate in some way the expression or at least one activity of the proteins and nucleic acids of the invention, such as the protein having the amino acid sequence of SEQ ID NO: 2. For osteoporosis, a disease characterized by abnormal bone density, the therapeutic strategy comprises a treatment with the agent until normal bone mass compared to appropriate control groups is restored. Bone mass can be assessed by determining bone mineral density. Then the treatment can be switched to established regimens for the prevention of bone loss to avoid potential side effects of overshooting bone formation.

The agents of the present invention can be provided alone, or in combination, or in sequential combination with other agents that modulate a particular pathological process. As used herein, two agents are said to be administered in combination when the two agents are administered simultaneously or are administered independently in a fashion such that the agents will act at the same time. For example, the agents of the invention can be used in combination with estrogen replacement therapy in postmenopausal osteoporosis.

The agents of the present invention can be administered via parenteral, subcutaneous, intravenous, intramuscular, intraperitoneal, transdermal, or buccal routes. For example, an agent may be administered locally to a site of injury via microinfusion. Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more agents that modulate expression or at least one activity of the proteins of the invention. While individual needs vary, determination of optimal ranges of effective amounts of each component is within the skill of the art. Typical dosages comprise 1.0 pg/kg to 100 mg/kg body weight. The preferred dosages for systemic administration comprise 100.0 ng/kg to 100.0 mg/kg body weight. The preferred dosages for direct administration to a site via microinfusion comprise 1 ng/kg to 1 mg/kg body weight.

In addition to the pharmacologically active agent, the compositions of the present invention may contain suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol and dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient. Suitable formulations for oral administration include hard or soft gelatin capsules, pills, tablets, including coated tablets, elixirs, suspensions, syrups or inhalations and controlled release forms thereof.

In practicing the methods of this invention, the agents of this invention may be used alone or in combination, or in combination with other therapeutic or diagnostic agents. In certain preferred embodiments, the compounds of this invention may be co-administered along with other compounds typically prescribed for these conditions according to generally accepted medical practice, such as anti-inflammatory agents, anticoagulants, antithrombotics, including platelet aggregation inhibitors, tissue plasminogen activators, urokinase, prourokinase, streptokinase, aspirin and heparin. The compounds of this invention can be utilized in vivo, ordinarily in mammals, such as humans, sheep, horses, cattle, pigs, dogs, cats, rats and mice, or in vitro. The invention is particularly useful in the treatment of human subjects.

Methods to Identify Agents that Modulate Expression

Another embodiment of the present invention provides methods for identifying agents that modulate the expression of a nucleic acid encoding a protein of the invention such as a protein having the amino acid sequence of SEQ ID NO: 2. Such assays may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, cell lines that contain reporter gene fusions between the open reading frame defined by nucleotides 319-1530 of SEQ ID NO: 1 and/or the 5′ and/or 3′ regulatory elements and any assayable fusion partner may be prepared. Numerous assayable fusion partners are known and readily available including the firefly luciferase gene and the gene encoding chloramphenicol acetyltransferase (Alam et al. (1990) Anal. Biochem. 188, 245-254). Cell lines containing the reporter gene fusions are then exposed to the agent to be tested under appropriate conditions and time. Differential expression of the reporter gene between samples exposed to the agent and control samples identifies agents that modulate the expression of a nucleic acid of the invention.

Additional assay formats may be used to monitor the ability of the agent to modulate the expression of a nucleic acid encoding a protein of the invention, such as the protein having SEQ ID NO: 2. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al. (1989) Molecular Cloning—A Laboratory Manual, Cold Spring Harbor Laboratory Press).

Probes to detect differences in RNA expression levels between cells exposed to the agent and control cells may be prepared from the nucleic acids of the invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementation that should exist between two nucleic acid strands in order to form a hybrid. Stringency should be chosen to maximize the difference in stability between the probe:target hybrid and probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al. (1989) Molecular Cloning—A Laboratory Manual, Cold Spring Harbor Laboratory Press or Ausubel et al. (1995) Current Protocols in Molecular Biology, Greene Publishing Co.

Hybridization conditions are modified using known methods, such as those described by Sambrook et al. and Ausubel et al as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The glass wafer can then be exposed to total cellular RNA or polyA RNA from a sample under conditions in which the affixed sequences will specifically hybridize. Such solid supports and hybridization methods are widely available, for example, those disclosed in WO 95/11755. By examining for the ability of a given probe to specifically hybridize to an RNA sample from an untreated cell population and from a cell population exposed to the agent, agents which up or down regulate the expression of a nucleic acid encoding the protein having the sequence of SEQ ID NO: 2 are identified.

Hybridization for qualitative and quantitative analysis of mRNA may also be carried out by using a RNase Protection Assay (i.e., RPA, see Ma et al. (1996) Methods 10, 273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45EC overnight in a buffer comprising 80% formamide, 40 mM Pipes (pH 6.4), 0.4 M NaCl and 1 mM EDTA. The resulting hybrids are then digested in a buffer comprising 40 μg/ml ribonuclease A and 2 μg/ml ribonuclease. After deactivation and extraction of extraneous proteins, the samples are loaded onto urea/polyacrylamide gels for analysis.

In another assay format, cells or cell lines are first identified which express the gene products of the invention physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines would be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag or other detectable marker. Such a process is well known in the art (see Sambrook et al. (1989) Molecular Cloning—A Laboratory Manual, Cold Spring Harbor Laboratory Press).

Cells or cell lines transduced or transfected as outlined above are then contacted with agents under appropriate conditions; for example, the agent in a pharmaceutically acceptable excipient is contacted with cells in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37EC. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the agent-contacted sample compared to the control will be used to distinguish the effectiveness of the agent.

Methods to Identify Agents that Modulate Activity

The present invention provides methods for identifying agents that modulate at least one activity of a protein of SEQ ID NO: 2. Such methods or assays may utilize any means of monitoring or detecting the desired activity.

In one format, the specific activity of a protein of the invention, normalized to a standard unit, between a cell population that has been exposed to the agent to be tested compared to an unexposed control cell population may be assayed. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes can be prepared by immunizing suitable mammalian hosts utilizing appropriate immunization protocols using the proteins of the invention or antigen-containing fragments thereof. To enhance immunogenicity, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) Biotechnology 24, 524-526 or modifications which effect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies may be recovered from the culture supernatant or from the ascites supernatant. Fragments of the monoclonal antibodies or the polyclonal antisera that contain the immunologically significant portion can be used as antagonists, as well as the intact antibodies. Use of immunologically reactive fragments, such as Fab or Fab′ fragments, is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin.

Antibody regions that bind specifically to the desired regions of the protein can also be produced in the context of chimeras with multiple species origin, for instance, humanized antibodies. The antibody can therefore be a humanized antibody or human a antibody, as described in U.S. Pat. No. 5,585,089 or Riechmann et al. (1988) Nature 332, 323-327.

Agents that are assayed in the above method can be randomly selected or rationally selected or designed. As used herein, an agent is said to be randomly selected when the agent is chosen randomly without considering the specific sequences involved in the association of the a protein of the invention alone or with its associated substrates, ligands, binding partners, etc. An example of randomly selected agents is the use a chemical library or a peptide combinatorial library, or a growth broth of an organism.

As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies or fragments thereof that bind to a protein of SEQ ID NO: 2. Antibody agents can be obtained by immunization of suitable mammalian subjects with peptides, containing as antigenic regions, those portions of the protein intended to be targeted by the antibodies.

In yet another class of agents, the present invention includes peptide mimetics that mimic the three-dimensional structure of the protein of SEQ ID NO: 2. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others.

In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

In another form, peptide analogs are commonly used in the pharmaceutical industry as non-peptide drugs with properties analogous to those of the template peptide. These types of non-peptide compounds are also referred to as peptide mimetics or peptidomimetics (Fauchere (1986) Adv. Drug Res. 15, 29-69; Veber & Freidinger (1985) Trends Neurosci. 8, 392-396; Evans et al. (1987) J. Med. Chem. 30, 1229-1239 which are incorporated herein by reference) and are usually developed with the aid of computerized molecular modeling.

Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage by methods known in the art.

Labeling of peptide mimetics usually involves covalent attachment of one or more labels, directly or through a spacer (e.g., an amide group), to non-interfering positions on the peptide mimetic that are predicted by quantitative structure-activity data and molecular modeling. Such non-interfering positions generally are positions that do not form direct contacts with the macromolecules to which the peptide mimetic binds to produce the therapeutic effect. Derivitization (e.g., labeling) of peptide mimetics should not substantially interfere with the desired biological or pharmacological activity of the peptide mimetic.

The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al. (1991) Proc. Natl. Acad. Sci. USA 88, 9578-9582) and using the phage display method. The two hybrid method detects protein-protein interactions in yeast (Fields et al. (1989) Nature 340, 245-246). The phage display method detects the interaction between an immobilized protein and a protein that is expressed on the surface of phages such as lambda and M13 (Amberg et al. (1993) Strategies 6, 2-4; Hogrefe et al. (1993) Gene 128, 119-126). These methods allow positive and negative selection for protein-protein interactions and the identification of the sequences that determine these interactions.

Diagnostic Methods and Agents

As described above, expression of the proteins and nucleic acids of the invention may be used as a diagnostic marker for the prediction or identification of the differentiation state of a sample comprising precursor stem cells. In some embodiments, the tissue sample is a bone biopsy. For instance, a tissue sample may be assayed by any of the methods described above, and expression levels of the proteins or nucleic acids of the invention may be compared to the expression levels found in undifferentiated precursor stem cells and/or precursor stem cells induced to differentiate into osteoblasts and/or precursor stem cells induced to differentiate into a cell type other than an osteoblast. Such methods may be used to diagnose or identify conditions characterized by abnormal bone deposition, reabsorption and/or abnormal rates of osteoblast differentiation.

Those skilled in the art will appreciate that a wide variety of conditions are associated with abnormal bone deposition or loss. Such conditions include, but are not limited to, osteoporosis, osteopenia, osteodystrophy, and various other osteopathic conditions. The methods of the present invention will be particularly useful in diagnosing or monitoring the treatment of conditions such as postmenopausal osteoporosis (PMO), glucocorticoid-induced osteoporosis (GIO), and male osteoporosis. Agents which modulate expression of the nucleic acids or proteins of the invention will be useful in treatment of these conditions.

In some preferred embodiments, the present invention may be used to diagnose and/or monitor the treatment of drug-induced abnormalities in bone formation or loss. For example, at present a combination of cyclosporine with prednisone is given to patients who have received an organ transplant in order to suppress tissue rejection. The combination causes rapid bone loss in a manner different than that observed with prednisone alone (such as elevated level of serum osteocalcin and vitamin D in patients treated with cyclosporine but not in patients treated with prednisone). Other drugs are also known to effect bone formation or loss. The anticonvulsant drugs diphenylhydantoin, phenobarbital and carbamazepine, and combination of these drugs, cause alterations in calcium metabolism. A decrease in bone density is observed in patients taking anticonvulsant drugs. Although heparin is an effective therapy for thromboembolic disorders, increased incidences of osteoporotic fractures have been reported in patients with heparin therapy hence the present invention will be useful to monitor patients undergoing heparin treatment.

Other embodiments of the present invention allow the diagnosis and/or monitoring of the treatment of other conditions that involve altered bone metabolism. For example, idiopathic juvenile osteoporosis (IJO) is a generalized decrease in mineralized bone in the absence of rickets or excessive bone resorption and typically occurs in children before the onset of puberty. In addition, thyroid diseases have been linked to bone loss. A decrease in bone mass has been shown in patients with thyrotoxicosis causing these individuals to be at increased risk of having fractures. These individuals also sustain fractures at an earlier age than individuals who have never been thyrotoxic.

Another situation in which the present invention will be useful is the diagnosis and/or monitoring of the treatment of skeletal disease linked to breast cancer. Breast cancer frequently metastasizes to the skeleton and about 70% of patients with advanced cancer develop symptomatic skeletal disease. Moreover, the anti-cancer treatments presently in use have been shown to lead to early menopause and bone loss when given to premenopausal women.

The present invention will be useful in diagnosing and/or monitoring the treatment of chronic anemia associated with abnormal bone formation or loss. Homozygous beta-thalassemia is usually described as an example of chronic anemia predisposing to osteoporosis. Patients with thalassemia have expansion of bone marrow space with thinning of the adjacent trabeculae.

Other conditions in which the present invention will find application are: Fanconi syndrome where osteomalacia is a common feature; fibrous dysplasia, McCune-Albright syndrome refers to patients with fibrous dysplasia with a sporadic, developmental disorder characterized by a unifocal or multifocal expanding fibrous lesion of bone-forming mesenchyme that often results in pain, fracture or deformity; osteogenesis imperfecta (OI, also called brittle bone disease) is associated with recurrent fractures and skeletal deformity, various skeletal dysplasias i.e., osteochondroplasia which is characterized by abnormal development of cartilage and/or bone and other diseases such as achodroplasia, mucopolysacchaidoses, dysostosis and ischemic bone diseases.

The present invention will be particularly useful by providing a marker that may be used as a marker of bone turnover to determine osteoporosis. The present invention may also be used in vitro in assays or treatments as a marker of osteoblast differentiation and proliferation.

Prognostic uses

As described above, the nucleic acids and proteins of the invention and their expression may also be used as markers for the monitoring of disease progression, such as osteoporosis. For instance, a tissue sample may be assayed by any of the methods described above, and the expression levels for the protein may be compared to the expression levels found in undifferentiated precursor stem cells and/or precursor stem cells induced to differentiate into osteoblasts and/or precursor stem cells induced to differentiate into a cell type other than an osteoblast and/or osteoblasts.

Expression or activity the proteins and nucleic acids of the invention, such as the protein having the amino acid sequence of SEQ ID NO: 2, may also be used to track or predict the progress or efficacy of a treatment regime in a patient. For instance, a patient's progress or response to a given drug may be monitored by measuring gene expression of the proteins of the invention in a tissue or cell sample after treatment or administration of the drug. The expression of the protein in the post-treatment sample may then be compared to gene expression from undifferentiated precursor stem cells and/or precursor stem cells induced to differentiate into osteoblasts and/or precursor stem cells induced to differentiate into a cell type other than an osteoblast and/or osteoblasts and/or from tissue or cells from the same patient before treatment.

Transgenic Animals

Transgenic animals containing mutant, knock-out or modified genes corresponding to the cDNA sequence of SEQ ID NO: 1, or the open reading frame encoding the polypeptide sequence of SEQ ID NO: 2 are also included in the invention. Transgenic animals are genetically modified animals into which recombinant, exogenous or cloned genetic material has been experimentally transferred. Such genetic material is often referred to as a transgene. The nucleic acid sequence of the transgene, in this case a form of SEQ ID NO: 1 may be integrated either at a locus of a genome where that particular nucleic acid sequence is not otherwise normally found or at the normal locus for the transgene. The transgene may consist of nucleic acid sequences derived from the genome of the same species or of a different species than the species of the target animal.

The term “germ cell line transgenic animal” refers to a transgenic animal in which the genetic alteration or genetic information was introduced into a germ line cell, thereby conferring the ability of the transgenic animal to transfer the genetic information to offspring. If such offspring in fact possess some or all of that alteration or genetic information, then they too are transgenic animals.

The alteration or genetic information may be foreign to the species of animal to which the recipient belongs, foreign only to the particular individual recipient, or may be genetic information already possessed by the recipient. In the last case, the altered or introduced gene may be expressed differently than the native gene.

Transgenic animals can be produced by a variety of different methods including transfection, electroporation, microinjection, gene targeting in embryonic stem cells and recombinant viral and retroviral infection (see, e.g., U.S. Pat. Nos. 4,736,866 & 5,602,307; Mullins et al. (1993) Hypertension 22, 630-633; Brenin et al. (1997) Surg. Oncol. 6, 99-110; Tuan (1997) Recombinant Gene Expression Protocols, Methods in Molecular Biology, Humana Press).

A number of recombinant or transgenic mice have been produced, including those which express an activated oncogene sequence (U.S. Pat. No. 4,736,866); express simian SV40 T-antigen (U.S. Pat. No. 5,728,915); lack the expression of interferon regulatory factor 1 (IRF-1) (U.S. Pat. No. 5,731,490); exhibit dopaminergic dysfunction (U.S. Pat. No. 5,723,719); express at least one human gene which participates in blood pressure control (U.S. Pat. No. 5,731,489); display greater similarity to the conditions existing in naturally occurring Alzheimer's disease (U.S. Pat. No. 5,720,936); have a reduced capacity to mediate cellular adhesion (U.S. Pat. No. 5,602,307); possess a bovine growth hormone gene (Clutter et al. (1996) Genetics 143, 1753-1760); or are capable of generating a fully human antibody response (McCarthy (1997) Lancet 349, 405-406).

While mice and rats remain the animals of choice for most transgenic experimentation, in some instances it is preferable or even necessary to use alternative animal species. Transgenic procedures have been successfully utilized in a variety of non-murine animals, including sheep, goats, pigs, dogs, cats, monkeys, chimpanzees, hamsters, rabbits, cows and guinea pigs (see, e.g., Kim et al. (1997) Mol. Reprod. Dev. 46, 515-526; Houdebine (1995) Reprod. Nutr. Dev. 35, 609-617; Petters (1994) Reprod. Fertil. Dev. 6, 643-645; Schnieke et al. (1997) Science 278, 2130-2133; and Amoah (1997) J. Animal Science 75, 578-585).

The method of introduction of nucleic acid fragments into recombination competent mammalian cells can be by any method that favors co-transformation of multiple nucleic acid molecules. Detailed procedures for producing transgenic animals are readily available to one skilled in the art, including the disclosures in U.S. Pat. Nos. 5,489,743 & 5,602,307.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLES

### Example 1

Up-Regulation of Expression in hFSC

Human Fetal Stromal Cells (hFSC) were isolated from the bone marrow of a twenty-week human embryo. hFSCs are derived from a primary culture and represent a heterogeneous population of osteoprogenitor cells. hFSCs exhibit a high replicative capacity, with a doubling time of approximately twenty hours. hFSCs retain a spindle-shaped morphology and have a uniform attachment throughout subcultivation. hFSCs can be sub-cultured up to twelve passages while retaining both proliferative and osteogenic capability.

hFSCs used for READS analysis or Quantitative RT-PCR were cultured in Dulbecco's Modified Eagle Medium (DMEM)-high glucose or DMEM-low glucose plus 10% fetal bovine serum, respectively, at 37° C. in a humidified atmosphere containing 95% air and 5% carbon dioxide in the absence and presence of the indicated treatment. RNA was extracted from the cells at thirty minutes, three hours, six hours, twelve hours, twenty-four hours, forty-eight hours, three days, six days, twelve days and twenty-four days. When indicated, cells were contacted with either bone morphogenic protein-2 (BMP-2) at 300 ng/ml or transforming growth factor beta (TGF-β) at 1 ng/ml. Cells were incubated for the period of time indicated and harvested. Total cellular RNA was prepared from the hFSC described above. Synthesis of cDNA and differential display by READS analysis was performed as previously described in WO 97/05286 and in Prashar et al. (1996) Proc. Natl. Acad. Sci. USA 93, 659-663.

Individual cDNA fragments corresponding to nucleic acid molecules of SEQ ID NO: 1 were separated by denaturing polyacrylamide gel electrophoresis and visualized by autoradiography. Bands identified as having different expression levels in treated versus untreated hFSC were extracted from the display gels as described by Liang et al. (1995) Curr. Opin. Immunol. 7, 274-280), reamplified using the 5′ and 3′ primers, and subcloned into PCR-Script with high efficiency using the PCR-Script® cloning kit (Stratagene). Plasmids were sequenced by cycle sequencing on an ABI automated sequencer. Alternatively, bands were extracted (cored) from the display gels, PCR amplified and sequenced directly without subcloning.

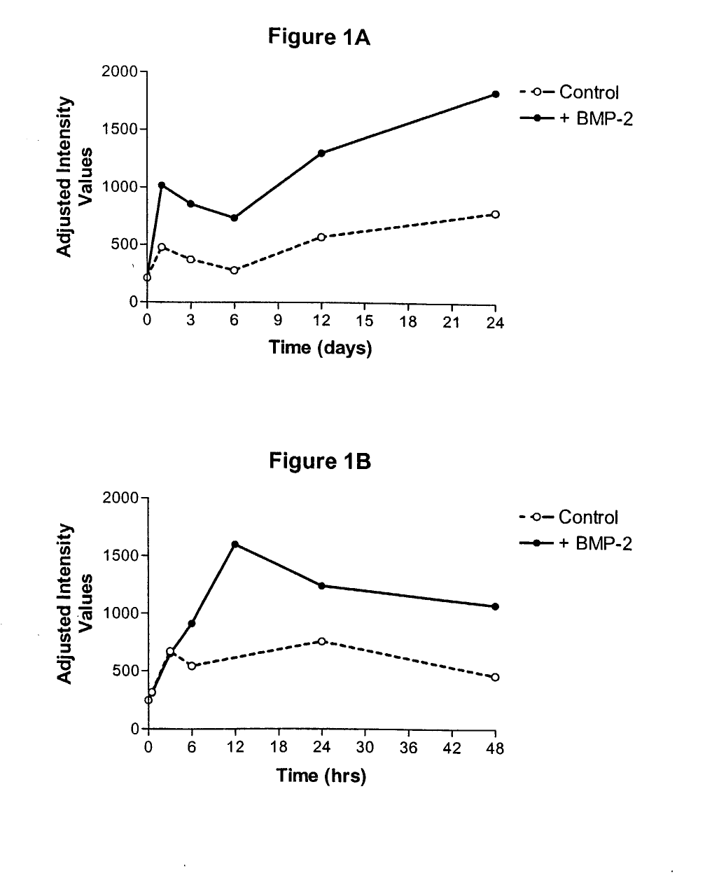

FIGS. 1A and B present a graphic depiction of the expression level of the target mRNA of SEQ ID NO: 1. READS analysis was performed on total RNA samples isolated from hFSC that were treated with BMP-2 (300 ng/ml of culture media) for twenty-four days in FIG. 1A and forty-eight hours in FIG. 1B. Time points for FIG. 1A were selected at one, three, six, twelve and twenty-four days post-initial treatment. Time points for FIG. 1B were selected at three, six, twelve, twenty-four and forty-eight hours.

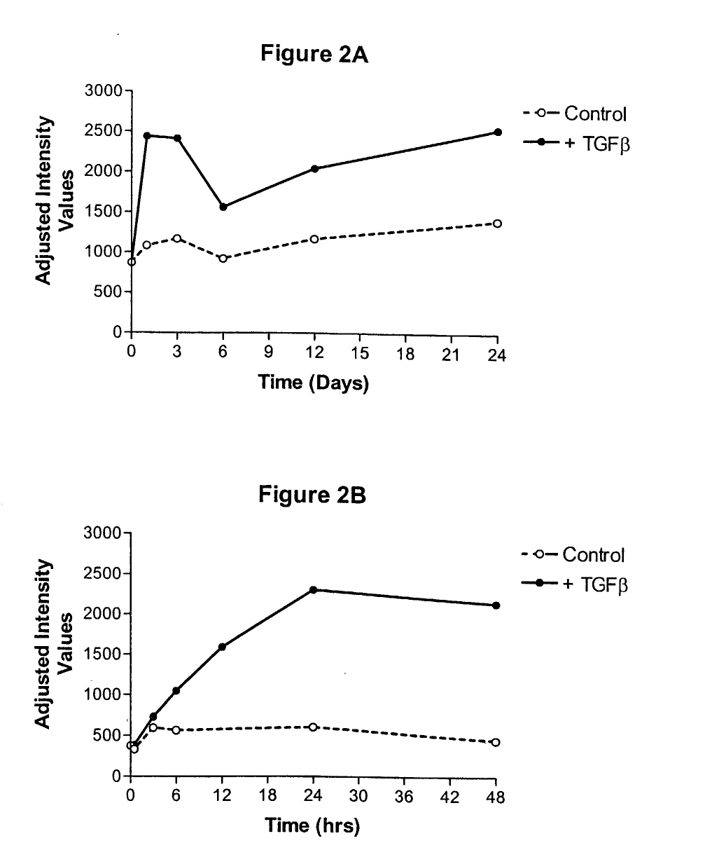

FIGS. 2A and B present a graphic depiction of the expression level of the target mRNA of SEQ ID NO: 1 in hFSC. READS analysis (as described above) was performed on total RNA samples isolated from hFSC that were either untreated controls or samples treated with osteogenic agent TGF-β (1 ng/ml of culture media) for twenty-four days in FIG. 2A and forty-eight hours in FIG. 2B. Time points for FIG. 2A were selected at one, three, six, twelve and twenty-four days post-initial treatment. Time points for FIG. 2B were selected at three, six, twelve, twenty-four and forty-eight hours.

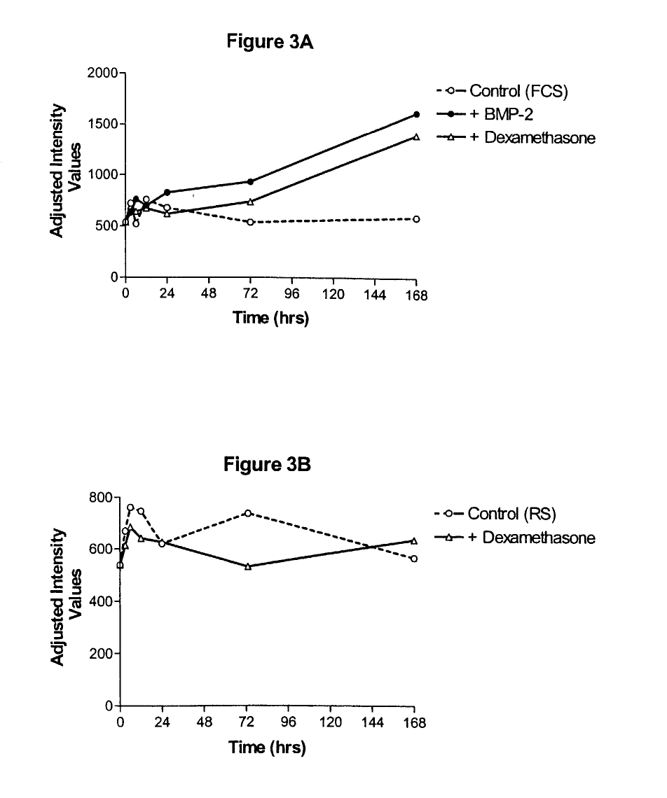

FIGS. 3A and 3B provide a graphical representation of the expression level of the target mRNA of SEQ ID NO: 1 in hMSC as assayed using READS gel analysis in response to treatment with osteogenic and adipogenic agents. In FIG. 3A, cells were cultured in a medium supplemented with 10% fetal calf serum with or without dexamethasone or BMP-2 for time period ranging from zero to seven days. In FIG. 3B, cells were cultured under adipogenic conditions in a medium containing 10% rabbit serum with or without addition of dexamethasone (100 nM) for the same time period.

Control cells received media only with no added osteogenic agent or adipogenic agent. Subsequent to READS gel analysis, the images of each gel were converted into electronic format and the intensities of each band of interest were calculated relative to the background autoradiographic intensity of each gel image. The corrected values are termed adjusted intensity values, which were plotted on the y-axis versus the time course of the experiment.

### Example 2

Cloning of Full Length Human Gene

The full length cDNA comprising SEQ ID NO: 1 was obtained by the solution hybridization method. Briefly, a gene-specific oligonucleotide was designed based on the sequence of an EST fragment identified by READS analysis (Prashar et al. (1996) Proc. Natl. Acad. Sci. USA 93, 659-663) as being upregulated during stem cell differentiation. The oligonucleotide was labeled with biotin and used to hybridize with 5 μg of single strand plasmid DNA (cDNA recombinants) from a human resting mast cell library following the procedures from the Gene Trapper kit (Life Technologies). The hybridized cDNA was separated by streptavidin-conjugated beads and eluted by Tris-EDTA buffer. The eluted cDNA was converted to double strand plasmid DNA and used to transform E. coli cells (DH5α). Clones were screened by PCR using gene specific primers designed from the EST sequence to identify positive clones. After positive selection, the cDNA clone was subjected to DNA sequence.

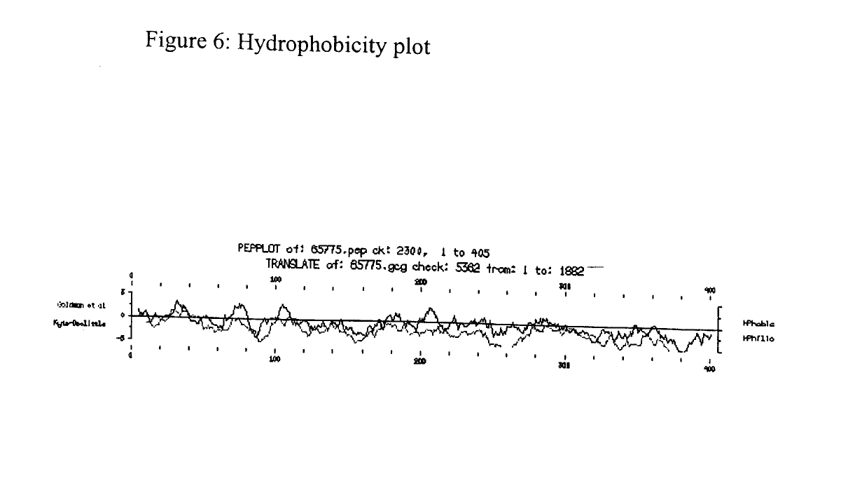

The nucleotide sequences of the full-length human cDNA corresponding to the differentially regulated mRNA detected above is set forth in SEQ ID NO: 1. The cDNA comprises 1882 base pairs, with an open reading frame at nucleotides 319-1533 encoding a protein of 405 amino acids (nucleotides 319-1530 without the stop codon). The amino acid sequence corresponding to the encoded protein is set forth in SEQ ID NO: 2. FIG. 6 displays the results of a hydrophobicity analysis of the polypeptide of SEQ ID NO: 2 using the methods of Kyte & Doolottle (1982) J. Mol. Biol. 157, 105-132.

### Example 3

Quantitative RT-PCR Analysis of Expression in hFSC and hMSC

Both hFSC and hMSC were used for this study as in the READS experiments. Briefly, PCR primers and TaqMan probes were designed using the DNA sequences provided by sequence analysis of the nucleic acid molecule of SEQ ID NO: 1. Experimental conditions were as follows: hFSC were cultured in vitro and were left untreated for up to twenty-four days, or were treated with the osteogenic agents TGF-β (1 ng/ml of culture media) or BMP-2 (300 ng/ml) for the same time period.

Cells in each of the treatment groups were harvested at various time points after addition of TGF-β or BMP-2. Total RNA was isolated from the cells using Trizol® and the RNA was quantitated using a spectrophotometer set at 260 nm. Ten ng of total RNA was assayed in duplicate using the TaqMan® assay (Perkin-Elmer) in biplex format where each target gene in each RNA sample was assayed versus a reference mRNA which was shown previously to be constitutively expressed and not regulated by any of the osteogenic treatments. The Ct values of the target and reference gene were analyzed and the delta Ct values were calculated for each RNA sample. Fold change (expressed as relative expression) was plotted versus the time course of the experiment. Expression was relative to the delta Ct value (Target Ct minus Reference Ct) for t=0 which was set to a value of 1.0.

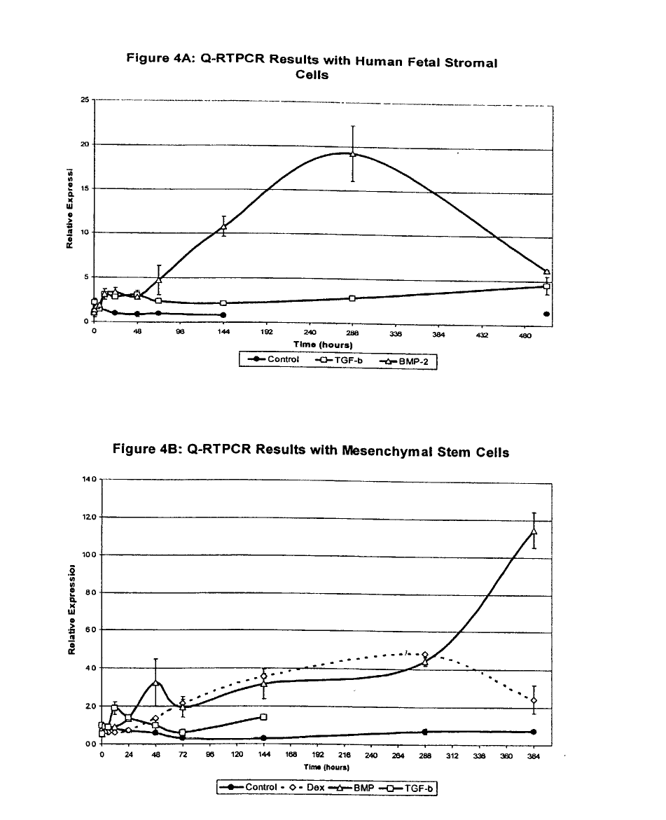

FIG. 4 shows expression levels of the target mRNA of SEQ ID NO: 1 in hFSC (4A) and in hMSC (4B) as assayed by quantitative RT-PCR. In FIG. 4A, cells were cultured using mineralization conditions in the absence (control, closed circles with solid line) or presence of either 1 ng/ml TGF-β1 (open squares) or 300 ng/ml of BMP-2 (open triangles) for time periods up to 504 hours (21 days). In FIG. 4B, hMSC were cultured in the absence and presence of either (1 ng/ml) TGF-β1, (300 ng/ml) BMP-2 or (100 nM) dexamethasone for time periods up to 384 hours (16 days).

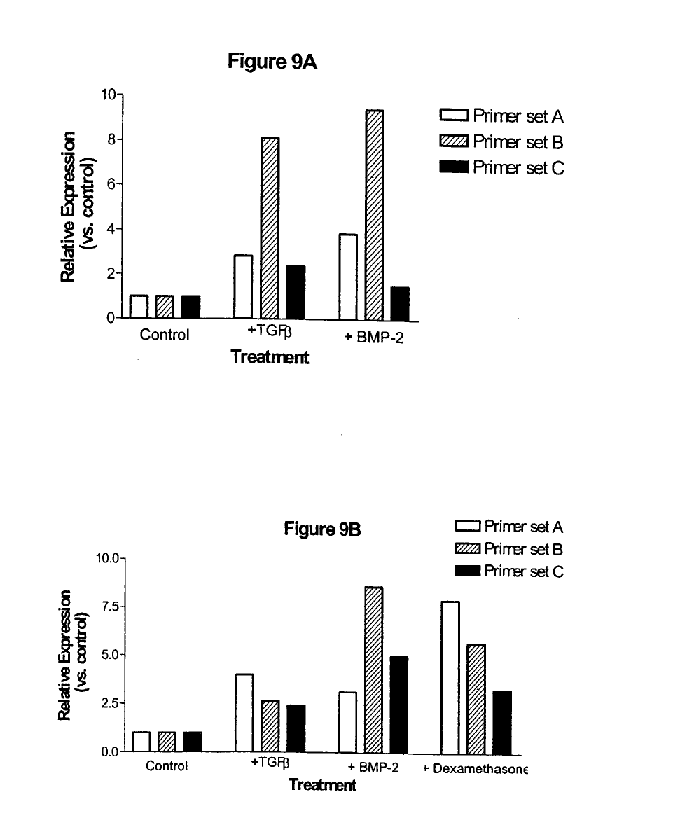

FIG. 9 shows a quantitative RT-PCR experiment where different primer sets were used that can differentiate PCR amplification between the wild-type cytokine type-1 CRLP-1 precursor (GenBank Accession No. AF073515) the the splice variant of this gene (SEQ ID NO: 1). Both hFSC (A) and hMSC (B) were used which were stimulated with the osteogenic agents shown for 24 hours or 48 hours, respectively. Primer set A consisted of a set of PCR primers directed against the known 5′ most exon of AF073515. Primer sets B and C consisted of PCR primer sets that would specifically support the amplification of SEQ ID NO: 1 and not of AF073515. Primer set B was composed of a primer set where the downstream primer was designed at the exon/exon junction (splice site) between the 5′ novel exon in SEQ ID NO: 1 and the immediate 3′ exon, whereas the upstream primer partner consisted of unique 5′ exon sequence in SEQ ID NO: 1. Primer set C consisted of a PCR primer set specific for the unique 5′ exon in SEQ ID NO: 1. The relative expression values are provided on the ordinate axis. The expression levels for the control (no osteogenic treatment) was set to 1.0.

### Example 4

Expression in Human Tissues

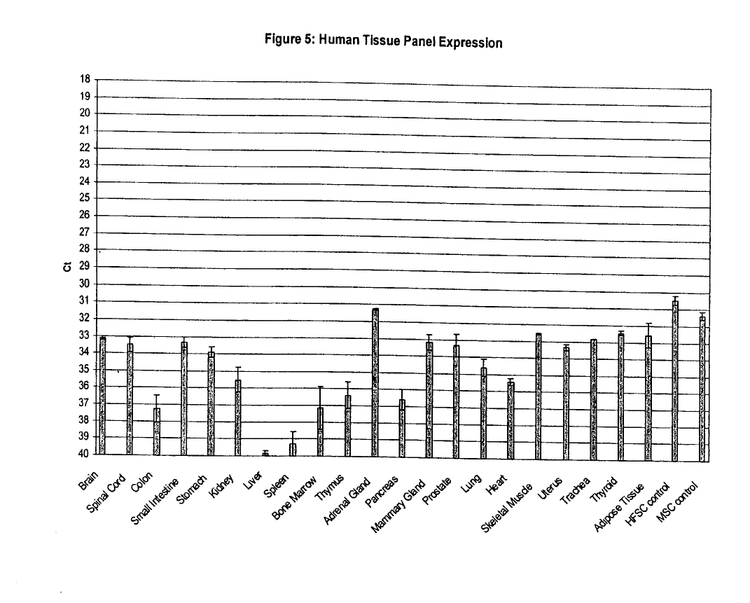

The tissue distribution of mRNA encoding the 65775 gene (SEQ ID NO: 1) was analyzed by quantitative PCR expression analysis of RNA isolated from various tissues. RNA was isolated from human kidney, spinal cord, adrenal gland, adipose tissue, heart, skeletal tissue, colon, pancreas, liver, prostate, thyroid, brain, stomach, small intestine, bone marrow, thymus, spleen, lung, uterus, mammary gland and trachea using standard procedures. PCR expression analysis was also performed using primers derived from the 65775 sequence using AmpliTaq® PCR amplification kits (Perkin Elmer). The presence of variable levels of mRNA encoding SEQ ID NO: 2 was detected in several tissues other than hFSC and hMSC (FIG. 5). mRNA expression was most abundant in the adrenal gland, skeletal muscle and thyroid. Detectable lower levels were observed in all other tissues tested.

FIG. 5 shows expression levels, depicted as Ct values, of the target mRNA of SEQ ID NO: 1 in various human tissues as assayed using TaqMan quantitative RT-PCR methods (described above). The Ct values are displayed on the y axis whereas the tissue panel utilized in the assay is provided on the x axis. Expression levels of the target mRNA in resting hFSC (HFSC control) and human mesenchymal stem cells (MSC control) is also provided

### Example 5

Northern Blot Analysis

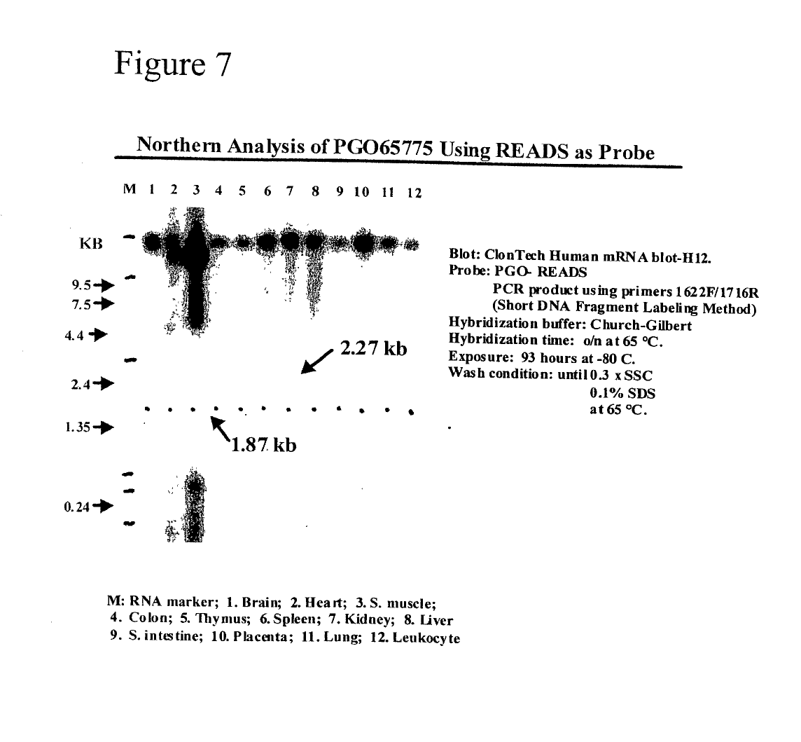

FIG. 7 shows a Northern blot in which the expression level of SEQ ID NO: 1 was measured in several normal human tissues including brain, heart, skeletal muscle, colon, thymus, spleen, kidney, liver, small intestine, placenta, lung and in leukocytes (ClonTech human mRNA blot-H12) as well as induced stem cells. RNA markers are present on the left side of the blot. Briefly, a cDNA clone corresponding to SEQ ID NO: 1, was radiolabeled using random primer labeling technology and the resulting probe was hybridized onto the blot during a sixteen hour incubation at 42° C. in a 50% formamide hybridization solution. After the hybridization, the blot was washed in 0.3×SSC, 0.1% SDS at 65° C. for up to two hours. After washing, the blot was exposed to film for a period of twenty-four hours at −80° C. prior to development to obtain the figure shown.

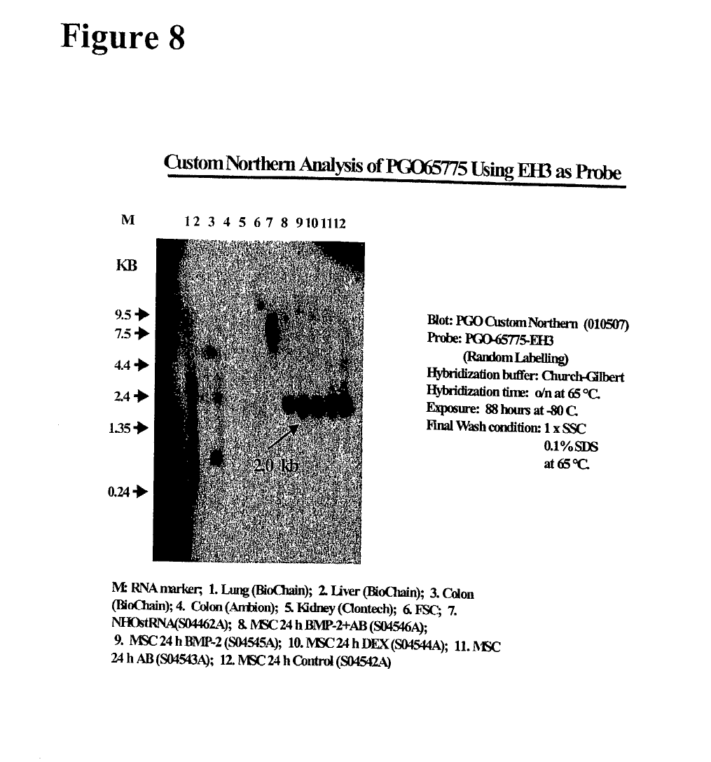

FIG. 8 shows the resulting autorad of a Northern blot in which the expression level of SEQ ID NO: 1 was measured in human tissues as well as in HFSC and hMSC either resting or treated with osteogenic agents. A radiolabeled probe corresponding to SEQ ID NO: 1 was constructed as described above and was hybridized onto the blot in Church-Gilbert solution for sixteen hours at 65° C. After hybridization, the blot was washed in 1.0×SSC/0.1% SDS at 65C for 2 hours before exposure to X-ray film.

### Example 6

Drug Screening Assays

Candidate agents and compounds will be screened for their ability to modulate the expression levels and/or activities of the gene comprising SEQ ID NO: 1 and identified as being involved in the differentiation of precursor stem cells into osteoblasts by any technique known to those skilled in the art including those assays described above. In some preferred embodiments, the assay of gene expression level may be conducted using real time PCR. Real time PCR detection may be accomplished by the use of the ABI Prism 7700 Sequence Detection System. The 7700 measures the fluorescence intensity of the sample each cycle and is able to detect the presence of specific amplicons within the PCR reaction. Each sample is assayed for the level of 65775 gene expression identified as being involved in the differentiation of precursor cells into osteoblasts.

The expression level of a control gene, for example GAPDH, may be used to normalize the expression levels. Suitable primers for the candidate genes may be selected using techniques well known to those skilled in the art. These primers may be used in conjunction with SYBR green (Molecular Probes), a nonspecific double stranded DNA dye, to measure the expression level mRNA corresponding to the 76032 gene, which will typically be normalized to the GAPDH level in each sample.

Normalized expression levels from cells exposed to the agent are then compared to the normalized expression levels in control cells. Agents that modulate the expression of the protein of this invention may be further tested as drug candidates in appropriate in vitro and in vivo models.

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety.

We claim:

1. An isolated nucleic acid molecule selected from the group consisting of:
(a) an isolated nucleic acid molecule comprising the nucleotide sequence of SEQ ID NO: 1;
(b) an isolated nucleic acid molecule encoding a polypeptide comprising the amino acid sequence of SEQ ID NO: 2;
(c) an isolated nucleic acid molecule that encodes a polypeptide fragment of at least 364 amino acids of SEQ ID NO: 2;
(d) an isolated nucleic acid molecule that exhibits at least 55% identity over the entire contiguous nucleotide sequence of SEQ ID NO: 1;
(e) an isolated nucleic acid molecule that exhibits at least 83% identity over the entire contiguous sequence of nucleotides 319-1533 of SEQ ID NO: 1; and
(f) an isolated nucleic acid molecule that encodes a polypeptide that exhibits at least 90% identity over the entire contiguous amino acid sequence of SEQ ID NO: 2.

2. The isolated nucleic acid molecule of claim 1, wherein the nucleic acid molecule comprises nucleotides 319-1533 of SEQ ID NO: 1.

3. The isolated nucleic acid molecule of claim 1, wherein the nucleic acid molecule consists of nucleotides 319-1533 of SEQ ID NO: 1.

4. The isolated nucleic acid molecule of claim 1, wherein the nucleic acid molecule comprises nucleotides 319-1530 of SEQ ID NO: 1.

5. The isolated nucleic acid molecule of claim 1, wherein said nucleic acid molecule is operably linked to one or more expression control elements.

6. A vector comprising an isolated nucleic acid molecule of claim 1.

7. A host cell transformed to contain the nucleic acid molecule of claim 1.

8. A host cell comprising the vector of claim 6.

9. The host cell of claim 7, wherein said host is selected from the group consisting of prokaryotic host cells and eukaryotic host cells.

10. A method for producing a polypeptide comprising culturing a host cell transformed with the nucleic acid molecule of claim 1 under conditions in which the polypeptide encoded by said nucleic acid molecule is expressed.

11. The method of claim 10, wherein said host cell is selected from the group consisting of prokaryotic host cells and eukaryotic host cells.

12. An isolated polypeptide produced by the method of claim 10.

13. An isolated polypeptide selected from the group consisting of:
(a) an isolated polypeptide comprising the amino acid sequence of SEQ ID NO: 2;
(b) an isolated polypeptide comprising a fragment of at least 364 amino acids of SEQ ID NO: 2; and
(c) an isolated polypeptide exhibiting at least 90% identity over the entire contiguous amino acid sequence of SEQ ID NO: 2.

14. The isolated polypeptide of claim 13, wherein the polypeptide consists of SEQ ID NO: 2.

15. An isolated antibody that specifically binds to a polypeptide of claim 13.

16. An antibody of claim 15 wherein said antibody is monoclonal.

17. A method of screening for an agent that modulates the differentiation of a population of stem cells into osteoblast cells comprising:
(a) exposing a population of stem cells to the agent, and
(b) measuring expression or activity of a nucleic acid molecule of claim 1 or a polypeptide encoded by the nucleic acid of claim 1 following exposure to the agent, wherein a increase in the level of expression or activity is indicative of an agent capable of stimulating stem cells to differentiate into osteoblast cells.

18. A method of screening for an agent that increases bone density comprising:
(a) exposing a population of stem cells to the agent; and
(b) measuring expression or activity of a nucleic acid molecule of claim 1 or a polypeptide encoded by the nucleic acid of claim 1 following exposure to the agent, wherein a increase in the level of expression or activity is indicative of an agent capable increasing bone density.

19. A method of diagnosing a condition characterized by abnormal stem cell differentiation comprising detecting in a stem cell sample the level of expression or activity of a nucleic acid molecule of claim 1 or a polypeptide encoded by the nucleic acid of claim 1, wherein a decrease in expression or activity compared to control stem cells is indicative of a condition characterized by abnormal bone density.

20. A method of diagnosing a condition characterized by abnormal bone density comprising detecting in a stem cell sample the level of expression or activity of a nucleic acid molecule of claim 1 or a polypeptide encoded by the nucleic acid of claim 1, wherein a decrease in expression or activity compared to control stem cells is indicative of a condition characterized by abnormal bone density.

21. The method of claim 20 wherein the condition is osteoporosis.

22. A non-human transgenic animal comprising a nucleic acid molecule of claim 1.

23. A non-human transgenic animal that is engineered to not express a protein encoded by a nucleic acid molecule of claim 1.
